(19) United States
(12) Patent Application Publication
(10) Pub. No.: US 2014/0243799 A1
(43) Pub. Date: Aug. 28, 2014

(54) Percutaneous Instrument with Tapered Shaft

(71) Applicant: ETHICON ENDO-SURGERY, INC., Cincinnati, OH (US)

(72) Inventor: Shailendra K. Parihar, Mason, OH (US)

(73) Assignee: Ethicon Endo-Surgery, Inc., Cincinnati, OH (US)

(21) Appl. No.: 13/779,211

(22) Filed: Feb. 27, 2013

(51) Int. Cl. A61B 17/00

(52) U.S. Cl. CPC A61B 17/00
USPC 606/1

(57) ABSTRACT

A surgical instrument comprises an elongate shaft having a proximal end and a distal end. The shaft tapers distally. An actuator is connected to the proximal end of the elongate shaft. An end effector is adapted for in vivo attachment and detachment from the distal end of the elongate shaft connected to the distal end of the elongate shaft. The actuator may comprise a manual handle. The elongate shaft may comprise an outer tube and an inner rod positioned in the outer tube, with the outer tube comprising a wall thickness increasing proximally. The surgical instrument may further comprise an intermediate tube positioned between the outer tube and inner rod. The elongate shaft may be adapted for direct percutaneous insertion.

### BACKGROUND

The present invention relates in general to surgical devices and procedures, and more particularly to minimally invasive surgery.

Surgical procedures are often used to treat and cure a wide range of diseases, conditions, and injuries. Surgery often requires access to internal tissue through open surgical procedures or endoscopic surgical procedures. The term “endoscopic” refers to all types of minimally invasive surgical procedures including laparoscopic, arthroscopic, natural orifice intraluminal, and natural orifice transluminal procedures. Endoscopic surgery has numerous advantages compared to traditional open surgical procedures, including reduced trauma, faster recovery, reduced risk of infection, and reduced scarring. Endoscopic surgery is often performed with an insufflatory fluid present within the body cavity, such as carbon dioxide or saline, to provide adequate space to perform the intended surgical procedures. The insufflated cavity is generally under pressure and is sometimes referred to as being in a state of pneumoperitoneum. Surgical access devices are often used to facilitate surgical manipulation of internal tissue while maintaining pneumoperitoneum. For example, trocars are often used to provide a port through which endoscopic surgical instruments are passed. Trocars generally have an instrument seal, which prevents the insufflatory fluid from escaping while an instrument is positioned in the trocar.

While a variety of different minimally invasive surgical devices are known, no one has previously made or used the surgical devices and methods in accordance with the present invention.

### SUMMARY

In one embodiment, a surgical instrument comprises an elongate shaft having a proximal end and a distal end. The shaft tapers distally. An actuator is connected to the proximal end of the elongate shaft. An end effector is adapted for in vivo attachment and detachment from the distal end of the elongate shaft connected to the distal end of the elongate shaft. The actuator may comprise a manual handle. The elongate shaft may comprise an outer tube and an inner rod positioned in the outer tube, with the outer tube comprising a wall thickness increasing proximally. The surgical instrument may further comprise an intermediate tube positioned between the outer tube and inner rod. The elongate shaft may be adapted for direct percutaneous insertion.

In another embodiment, a surgical instrument comprises an elongate shaft having an outer tube and an inner rod positioned in the outer tube. The outer tube and inner rod each comprise a distal end and a proximal end. The outer tube comprises a wall thickness that tapers distally. An actuator is connected to the proximal ends of the outer tube and inner rod. An end effector is adapted for in vivo attachment and detachment from the distal ends of the outer tube and inner rod. The actuator may comprise a manual handle. The distal end of the inner rod may comprise a pointed tip. The elongate shaft may be adapted for direct percutaneous laparoscopic insertion. An intermediate tube may be positioned between the outer tube and an inner rod. The intermediate tube may comprise a proximal end connected to the actuator and a distal end adapted for in vivo attachment and detachment from the end effector.

In yet another embodiment, a surgical instrument is adapted for direct percutaneous insertion. An elongate shaft comprises a proximal end and a distal end. The elongate shaft has an area moment of inertia that increases towards the proximal end. An actuator is operably connected to the proximal end of the elongate shaft. An end effector is adapted for in vivo attachment to and detachment from the distal end the elongate shaft. The elongate shaft may comprise an outside diameter less than 3 mm.

### BRIEF DESCRIPTION OF DRAWINGS

While the specification concludes with claims which particularly point out and distinctly claim the invention, it is believed the invention will be better understood from the following description taken in conjunction with the accompanying drawings illustrating some non-limiting examples of the invention. Unless otherwise indicated, the figures are not necessarily drawn to scale, but rather to illustrate the principles of the invention.

FIG. 1 depicts a surgical instrument with end effector, shaft, and actuator;

FIG. 2 depicts an exploded view of the instrument of FIG. 1; and

FIG. 3 depicts an exploded view of the shaft of FIGS. 1-2.

### DETAILED DESCRIPTION

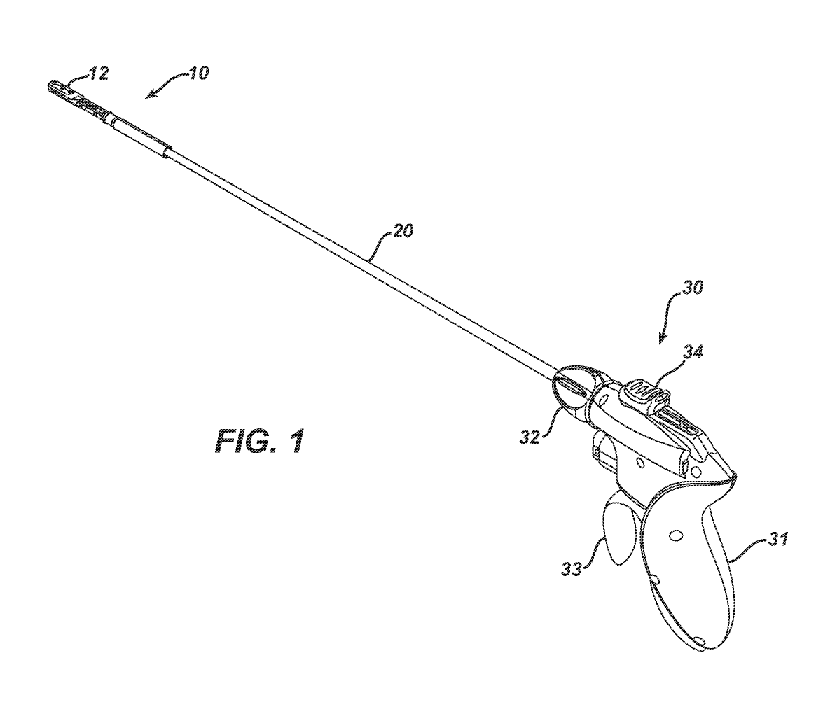

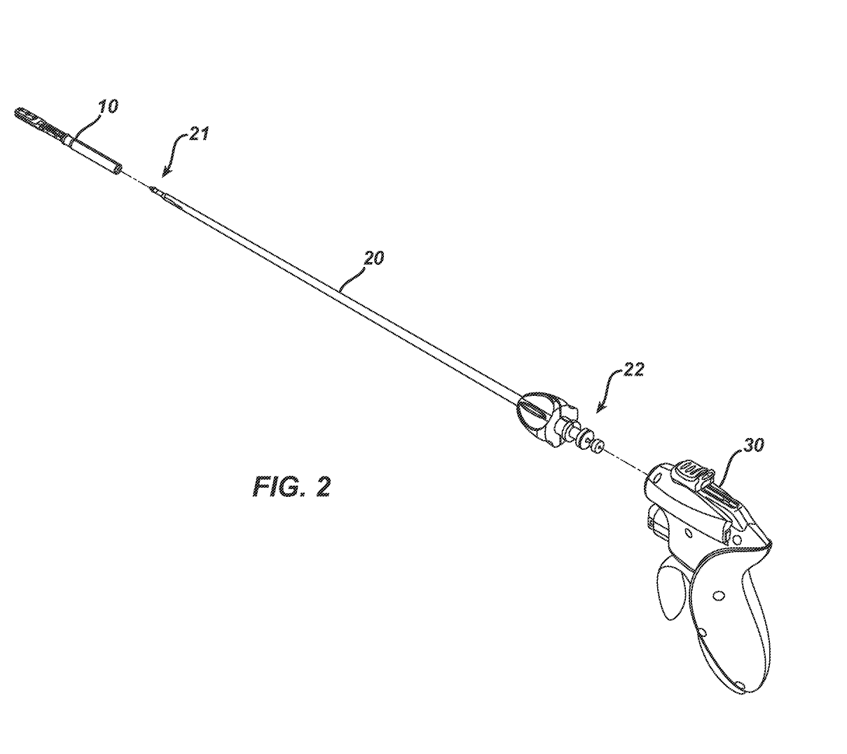

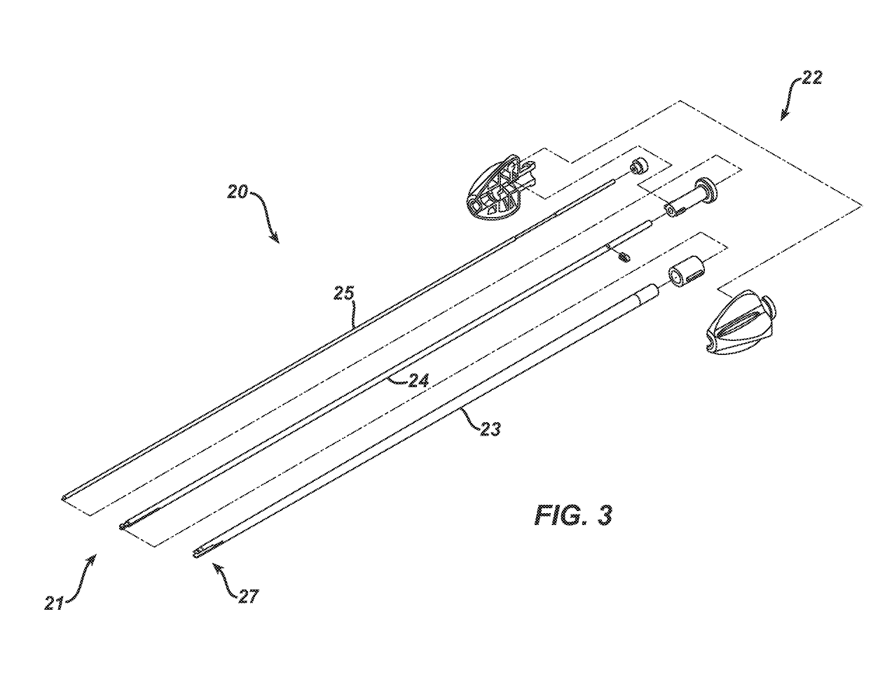

FIGS. 1-3 illustrate an example a laparoscopic surgical instrument. The elongate shaft (20) comprises a distal end (21) and a proximal end (22). The elongate shaft (20) may be rigid and adapted for insertion into a body cavity through an access device, such a trocar, or through direct percutaneous insertion without an access device. The elongate shaft (20) comprises two or more coaxially nested shafts operable to actuate the end effector (10). In this embodiment, the elongate shaft comprises an outer tube (23), an intermediate tube (24), and an inner rod (25) that can axially slide relative one another.

An actuator (30) is operably connected to the proximal ends (22) of the outer tube (23), an intermediate tube (24), and an inner rod (25). In this embodiment the actuator (30) is a manual pistol grip handle; however, a variety of other manual actuators could also be used, including a scissor grip handle, a syringe grip handle, endoscopic rotary knobs, and the like. The actuator (30) could also take the form of a robotic interface, such as an DAVINCI puck, a housing comprising gears or pulleys, servomechanisms, and the like.

The end effector (10) is adapted for in vivo attachment to and detachment from the elongate shaft (20). The attachment/detachment mechanism include mating features (27) that engage corresponding mating features of the end effector (10). The particular mating features are not significant to the present invention and may can include threads, collets, bayonets, and the like. Some non-limiting examples are disclosed in U.S. application Ser. Nos. 12/576,546 and 12/889,454, which are incorporated herein by reference.

The end effector (10) in this embodiment includes a pair of surgical jaws (12) have an opened position and closed position. In this embodiment, the jaws (12) are shown as a grasper; however, a variety of other tissue manipulating jaws could also be used, including dissectors, sheers, babcocks, forceps, staplers, clip appliers, and the like. Non-jawed end effectors (10) could also be employed such as hook knives, snares, retractors, and the like. In the case of end effectors that require energy, appropriate energy transmission mechanisms known in the art can be added. For instance, appropriate electrical connections can be added between the shaft (20) and end effector (10) to enable bi-polar forceps. Similarly, an ultrasonic transducer and waveguide can be added for the ultrasonic shears end effector.

During surgery, the distal end (21) of the shaft (20) is inserted into the body cavity, such as the abdomen, pelvis, thorax, etc. The end effector (10) as also introduced into the body cavity, typically through an access port. Optionally, the end effector (10) can be introduced with a separate loader, such as that disclosed in U.S. application Ser. No. 12/576,565. The end effector (10) is then attached in vivo to the shaft (20). Operation of the actuator (30) translates to operate the jaws, thus enabling a surgeon to manipulate tissue and perform minimally invasive surgical procedures.

The outer tube (23) tapers distally. In this embodiment such tapering is created by increasing the wall thickness towards the proximal end (22). The inner lumen of the outer tube (23) may have a uniform diameter along its length. In an alternative embodiment, such tapering can be achieve by adding longitudinal ribs towards the proximal end (22). In yet another embodiment, the outer tube (23) may be stepped. The tapering feature increases the area moment of inertia towards the proximal end (22) and thus increases the strength and decreases deflection of the shaft (20) when lateral forces are introduced on the end effector (10). For instance, retracting a heavy organ, such as a liver, can produce large loads on the surgical instrument that may excessively deflect or plastically deform the shaft (20). This is particular advantageous with very slender shafts (e.g., having an outside diameter less than 3 mm). For instance, the outside diameter may taper from about 2 mm at the distal end to about 5 mm at the proximal end. In applications involving direct percutaneous insertion, the tapering, and hence gradual dilation of tissue, can minimize trauma to the insertion wound.

Having shown and described various embodiments and examples of the present invention, further adaptations of the methods and devices described herein can be accomplished by appropriate modifications by one of ordinary skill in the art without departing from the scope of the present invention. Several of such potential modifications have been mentioned, and others will be apparent to those skilled in the art. For instance, the specific materials, dimensions, and the scale of drawings will be understood to be non-limiting examples. Accordingly, the scope of the present invention should be considered in terms of the following claims and is understood not to be limited to the details of structure, materials, or acts shown and described in the specification and drawings.

1. A surgical instrument, comprising:
an elongate shaft having a proximal end and a distal end, the shaft tapering distally;
an actuator connected to the proximal end of the elongate shaft; and
an end effector adapted for in vivo attachment and detachment from the distal end of the elongate shaft connected to the distal end of the elongate shaft.

2. The surgical instrument of claim 1, wherein the actuator comprises a manual handle.

3. The surgical instrument of claim 1, wherein the elongate shaft comprises outer tube and an inner rod positioned in the outer tube, the outer tube comprising a wall thickness increasing proximally.

4. The surgical instrument of claim 3, further comprising an intermediate tube positioned between the outer tube and inner rod.

5. The surgical instrument of claim 1, wherein the elongate shaft is adapted for direct percutaneous insertion.

6. A surgical instrument, comprising:
an elongate shaft comprising an outer tube and an inner rod positioned in the outer tube, the outer tube and inner rod each comprising a distal end and a proximal end, the outer tube comprising a wall thickness that tapers distally;
an actuator connected to the proximal ends of the outer tube and inner rod; and
an end effector adapted for in vivo attachment and detachment from the distal ends of the outer tube and inner rod.

7. The surgical instrument of claim 6, wherein the actuator comprises a manual handle.

8. The surgical instrument of claim 6, wherein the distal end of the inner rod comprises a pointed tip.

9. The surgical instrument of claim 6, wherein the elongate shaft is adapted for direct percutaneous insertion.

10. The surgical instrument of claim 6, further comprising an intermediate tube positioned between the outer tube and an inner rod, the intermediate tube comprising a proximal end connected to the actuator and a distal end adapted for in vivo attachment and detachment from the end effector.

11. A surgical instrument adapted for direct percutaneous insertion, comprising:
an elongate shaft comprising an proximal end and a distal end, the elongate shaft having an area moment of inertia increasing towards the proximal end;
an actuator operably connected to the proximal end of the elongate shaft; and
an end effector adapted for in vivo attachment to and detachment from the distal end the elongate shaft.

12. The surgical instrument of claim 11, wherein the elongate shaft comprises an outside diameter less than 3 mm. The surgical instrument of claim 11, wherein the elongate shaft comprises an outside diameter tapering from about 2 mm at the distal end to about 5 mm at the proximal end.
